(19) United States
(12) Patent Application Publication
(10) Pub. No.: US 2022/0047734 A1
(43) Pub. Date: Feb. 17, 2022

(54) AIRCRAFT DISINFECTING SYSTEM

(71) Applicant: HONEYWELL INTERNATIONAL INC., Charlotte, NC (US)

(72) Inventors: Peter Michalakos, Des Plaines, IL (US); Sunit Kumar Saxena, Bangalore (IN); Stephen Yates, Des Plaines, IL (US)

(73) Assignee: HONEYWELL INTERNATIONAL INC., Charlotte, NC (US)

(21) Appl. No.: 17/032,940

(22) Filed: Sep. 25, 2020

(30) Foreign Application Priority Data
Aug. 13, 2020 (IN) 202011034847

(51) Int. Cl. A61L 2/10; A61L 2/24

(52) U.S. Cl. CPC A61L 2/10; A61L 2/24; A61L 2202/14; A61L 2202/16; A61L 2202/25

(57) ABSTRACT

An aircraft disinfecting system. The system includes a trolley that can move along an aisle of an aircraft and an arm assembly coupled to the trolley. The arm assembly includes at least first and second arms that can extend over one or more seats on either side of the aisle along respective axes of extension. An electromagnetic radiation apparatus coupled to the trolley and the arm assembly for disinfecting, by electromagnetic irradiation, surfaces associated with the seats. The first and second arms are rotatable about the axes of extension, respectively.

### CROSS REFERENCE TO RELATED APPLICATION

This application claims priority to Indian Provisional Patent Application No. 202011034847, filed Aug. 13, 2020, the entire content of which is incorporated by reference herein.

### TECHNICAL FIELD

The present disclosure generally relates to apparatus and systems for disinfecting surfaces in an aircraft using electromagnetic radiation. Further, the present disclosure relates to systems for automated control of an apparatus for disinfecting aircraft surfaces.

### BACKGROUND

Infectious disease transmission among air travelers is a significant personal and public health concern. Common and potentially serious viral (e.g. Influenza, Covid-19), bacterial (e.g. Methicillin Resistant Staph aureus), and fungal pathogens are typically spread through the air and from mutually contacted surfaces, known as “fomites”. Commercial aircraft currently use extensive on-board air filtration and ultraviolet “C” band (UVC) (extrinsic to cabin compartment) technologies to decrease airborne microbes, yet disease transmission continues, suggesting cabin surfaces may play a role. The interior of an aircraft presents a challenge for UVC sanitization because there is limited room to maneuver and there are many surfaces. The performance of a UV disinfecting system is determined, at least in part, by the dosage of UV light, which is related to the incident angle and the inverse of distance squared. There is a further need to provide additional functionality in order to properly disinfect galleys, lavatories, floors in-between seat rows, and the cockpit.

Hence, it is desirable to provide systems that allow more consistent and more effective disinfection of aircraft surfaces throughout the interior of the cabin. Yet further, it would be desirable to provide more adaptable disinfecting systems using electromagnetic radiation. Furthermore, other desirable features and characteristics of the present invention will become apparent from the subsequent detailed description and the appended claims, taken in conjunction with the accompanying drawings and the foregoing technical field and background.

### SUMMARY

This summary is provided to describe select concepts in a simplified form that are further described in the Detailed Description. This summary is not intended to identify key or essential features of the claimed subject matter, nor is it intended to be used as an aid in determining the scope of the claimed subject matter.

An aircraft disinfecting system. The system includes a trolley that can move along an aisle of an aircraft and an arm assembly coupled to the trolley. The arm assembly includes at least first and second arms that can extend over one or more seats on either side of the aisle along respective axes of extension. An electromagnetic radiation apparatus coupled to the trolley and the arm assembly for disinfecting, by electromagnetic irradiation, surfaces associated with the seats. The first and second arms are rotatable about the axes of extension, respectively.

Furthermore, other desirable features and characteristics will become apparent from the subsequent detailed description and the appended claims, taken in conjunction with the accompanying drawings and the preceding background.

### BRIEF DESCRIPTION OF THE DRAWINGS

The present invention will hereinafter be described in conjunction with the following drawing figures, wherein like numerals denote like elements, and wherein:

FIG. 1 is a schematic diagram of an aircraft disinfecting system, in accordance with embodiments of the present disclosure;

FIG. 2 is a schematic diagram of another view of the aircraft disinfecting system of FIG. 1, in accordance with embodiments of the present disclosure; and

FIG. 3 is a schematic diagram of another aircraft disinfecting system, in accordance with embodiments of the present disclosure.

### DETAILED DESCRIPTION

The following detailed description is merely exemplary in nature and is not intended to limit the application and uses. Furthermore, there is no intention to be bound by any expressed or implied theory presented in the preceding technical field, background, summary, or the following detailed description. As used herein, the term “controller” refers to any hardware, software, firmware, electronic control component, processing logic, and/or processor device, individually or in any combination, including without limitation: application specific integrated circuit (ASIC), a field-programmable gate-array (FPGA), an electronic circuit, a processor (shared, dedicated, or group) and memory that executes one or more software or firmware programs, a combinational logic circuit, and/or other suitable components that provide the described functionality.

Embodiments of the present disclosure, particularly the controller, may be described herein in terms of functional and/or logical block components and various processing steps. It should be appreciated that such block components may be realized by any number of hardware, software, and/or firmware components configured to perform the specified functions. For example, an embodiment of the present disclosure may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments of the present disclosure may be practiced in conjunction with any number of systems, and that the systems described herein is merely exemplary embodiments of the present disclosure.

For the sake of brevity, conventional techniques related to signal processing, data transmission, signaling, control, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in an embodiment of the present disclosure.

In accordance with various embodiments, an aircraft disinfecting system includes articulated arms that are variously orientable, adjustable and extendable. In various embodiments, the arms are extendable to selectable lengths along an extension axis and are rotatable about the extension axis to allow control of location and angle of incidence of impingement of radiation onto aircraft cabin surfaces. In one embodiment, at least two arms (and optionally three arms) are provided including one arm in the front that can be used for the floor and tight passages (e.g. lavatory, cockpit, galley) and one or two arms extending sideways. In another embodiment, a tower from which the articulated arms extend can rotate so that the at least two articulated arms may reach in different directions than side-to-side. For example, the tower may rotate 90° and allow one arm to extend forward into the cabin without turning a trolley to which the tower is coupled.

In another embodiment, the arms can unfold or otherwise extend to lengths independently of each other. Additionally, or alternatively, the height of the arms can be adjusted and/or the height of the arms can be adjusted so as to be different from one another. In this way, it is possible to customize radiation application for non-symmetric cabin configurations (e.g. 2 seats on one side and 3 on the other; or 2 seats on one side, and 1 on the other). Further, the proximity of the radiation to the aircraft surface can be better controlled. In some embodiments, the arms are height adjustable, length adjustable and rotatable about the axis of extension to allow control of optimal distance from aircraft surfaces, coverage of aircraft surfaces and angle of incidence. Such adjustments may be motorized and controlled automatically or by a user through a user interface. Alternatively, the adjustments may be manually executed, optionally with the assistance of levers or knobs.

In one embodiment, which may be combined with the above embodiments, a combined or split configuration of each set of arms is contemplated. For example, X-wing or bi-level arms are disclosed whereby each articulated arm not only extends but splits into an upper arm assembly to reach overhead bins and passenger service unit (PSU), while a lower arm assembly separates to be closer to seats and floor. The cross-section of such arms may be in the form of the letter “X.”

In some embodiments, the aircraft disinfecting system is configured to apply ultraviolet (UV) radiation to surfaces of the interior of the aircraft. More than one wavelength of UV may be used. For example, 200-220 nm may be useable in the presence of humans (or at least humans not wearing protective clothing) because the radiation is less harmful and germicidal wavelengths of 250-270 nm may be used in the absence of humans (or at least humans wearing protective clothing). These may be user or system selectable depending on the application: for example, if there are other ground crew or maintainers in the cabin. In embodiments, light-emitting diodes (LEDs) are used as the light source with pulse-width modulation (duty cycle). High intensity pulses can be applied to specific areas that are at acute angles; whereas continuous radiation levels can be used at areas where the incident angle is around 90 degrees. The selection between intensity levels can be user or system selectable. The pulse frequency (the cyclical time period between neighboring pulses) of radiation also plays a role in disinfection efficiency. For example, virus disinfection rate can be controlled by varying the applied radiation pulse frequency.

In one embodiment, there are two arms oscillating up and down as the trolley proceeds down the aisle of the aircraft cabin. The arms may follow the contours of the interior surfaces such that the lights on the arms are at a desired distance away from the surfaces. The arms may be raised and lowered from their base at the trolley. For example, a cam may be coordinated with wheel speed to raise and lower the arms. The ratio between wheel speed and arm oscillation may be programmed based on aircraft model or airline seat pitch. In another embodiment, the oscillation of the arms is enabled by a sensor that is able to detect seat pitch. In a further embodiment, the arms pivot around the base of the joint (about the axis of extension) so that the angle of incidence can be perpendicular to the surface. Both the raising and lowering of the arms and the turning of the arms around the axis of extension may be performed automatically (through a cam or programmed motor controller). In one example, the light bank on the arms may be pointed down above a seat bottom, and tilt up as the arms get close to the seat backs and tray tables.

Accordingly, various embodiments of an aircraft disinfecting system are disclosed herein. In one embodiment, there is a UVC based sanitization system comprising extendable arms at least in a lateral (sideways) direction and optionally also in a front direction. The arms are rotatable about their axis of extension. The arms may be equipped with UVC sources in Germicidal (e.g. 254 nm) and Far UVC (200-220 nm) range. Radiation of a particular wavelength may be emitted based on the human occupancy of the space, which needs to be sanitized. Radiation may be emitted at varying intensity levels either in continuous mode or in pulsed mode. Human occupancy can be motion detected or manually set by the operator. High intensity pulses can be applied in those certain areas which are at acute angles; whereas continuous radiation levels can be applied at areas where incident angle is around 90 degrees. The pulse frequency may influence the disinfection efficiency. For example, if virus disinfection rate varies with applied pulse frequency, then pulse frequency, pulse width and pulse amplitude can be controlled with UV LEDs and suitable electronics control circuitry.

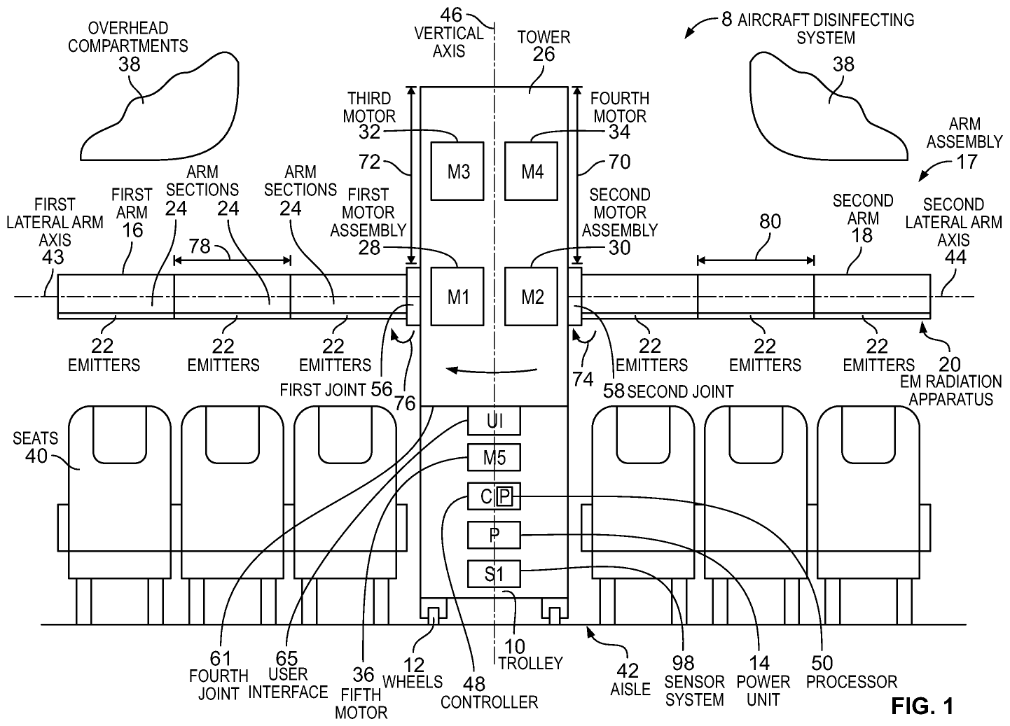

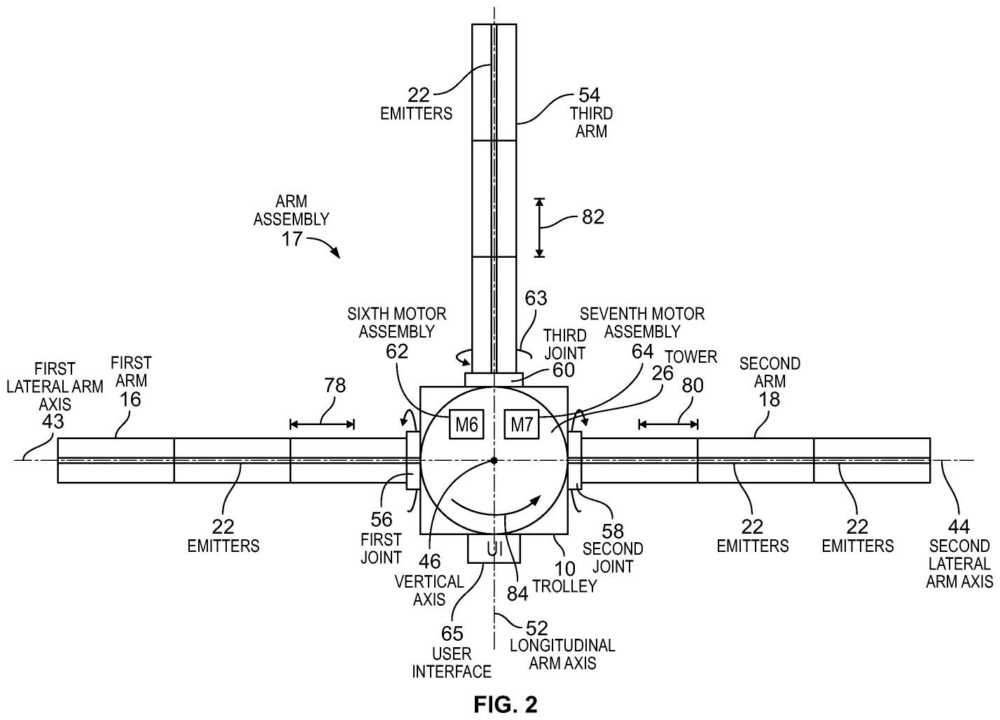

With reference to FIGS. 1 and 2, there is disclosed an aircraft disinfecting system 8 according to an exemplary embodiment of the present disclosure. The aircraft disinfecting system 8 includes a trolley 10, a tower 26 coupled to, and mounted above, the trolley 10 and an arm assembly 17. The trolley 10 may have a footprint similar to that of a standard food/beverage trolley used on an aircraft but may be of a greater height. The trolley 10 has wheels 12 to allow the trolley 10 to be manually pushed by an operative or motor driven under operative control along an aisle 42 of an aircraft.

The arm assembly 17 includes a first arm 16, a second arm 18 and (with reference to FIG. 2) a third arm 54. One of the first, second and third arms 16, 18, 54 may be omitted in some embodiments. Each of the first, second and third arms 16, 18, 54 include emitters 22 for emitting disinfecting radiation onto surfaces of an aircraft cabin. The first and second arms 16, 18 extend over one or more seats 40 on each side of the aisle 42. In the depicted embodiment, there are three seats on each side of the aisle 42 but other symmetric numbers and non-symmetric numbers of seats are possible. The first and second arms 16, 18 are connected to the tower 26 and are variably laterally extendable along respective lateral arm axes 43, 44. The first and second arms 16, 18 are able to be positioned above the aircraft seats 40 and below the overhead compartments 38 (or storage bins 38). The third arm is longitudinally extendable along the aisle 42 or along the longitudinal arm axis 52. As will be described further herein, the lateral extent of the first and second arms 16, 18 is adjustable. The longitudinal extent of the third arm 54 may also be adjustable. This adjustment of the lateral and longitudinal extent of the first to third arms 16, 18, 54 may be independent of one another. In this way, any one of the first to third arms 16, 18, 54 may be extended to a different length than the others. With respect to the first and second arms 16, 18, the different lengths accommodate different numbers of seats on each side of the aisle 42. Further, the height of the first and second arms 16, 18 is adjustable so as to be movable closer to the overhead compartments 38 and closer to the base of the seats 40. The height of the third arm 54 may also be adjustable. In this way, tray tables, seat backs (front side and rear side) and overhead compartments 38 may all be effectively sterilized by the first and second arms 16, 18. Yet further, areas located in front of the trolley 10 along the aisle 42 may be sterilized by the third arm 54 including toilets, galleys, cockpits, etc.

The first to third arms 16, 18, 54 may be retracted and stowed within, or substantially within, the footprint of the trolley 10 for storage and when maneuvering the trolley 10 into position and on/off the aircraft when the trolley 10 is not stored on the aircraft. The first to third arms 16, 18, 54 may be retractable and extendable independently of each other such that just one or two of the first to third arms 16, 18, 54 are in an extended state. For example, it may be desirable to operate with just the third arm 54 being extended and with the first and second arms 16, 18 being retracted.

The first, second and third arms 16, 18, 54 may be configured in a variety of ways. FIGS. 1 and 2 illustrate the first to third arms 16, 18, 54 as being made up of a plurality of arm sections 24 that are telescopically arranged with respect to each other. Each arm section 24 may have a respective emitter 22 that is electrically connected to allow independent activation of the emitters 22 depending on the number of sections 24 that are deployed. The arm sections 24 may have a longitudinal slot through which light from the emitters 22 radiates. The exemplary embodiment shows three arm sections 24 for each of the first to third arms 16, 18, 54 but other numbers of arm sections 24 are contemplated. Although a telescopic arrangement for the first to third arms 16, 18, 54 is depicted in the Figures, other configurations are possible including a folding frame attached to a scissors like extension/retraction mechanism, roll-up type extendable and retractable arms, etc.

The tower 26 is coupled atop the trolley 10. In one embodiment, the tower 26 is coupled to the trolley 10 by a fourth joint 61 that allows rotation of the tower 26 relative to the trolley 10 about a vertical axis 46 extending through the trolley 10 and the tower 26, as indicated by arrow 84. The first, second and third arms 16, 18, 54 are coupled to the tower 26 by respective first, second and third joints 56, 58, 60. The first, second and third joints 56, 58, 60 (or a subset of one or two thereof) allow rotation of the first and second arms 16, 18 about their respective first and second lateral arm axes 43, 44, as indicated by arrows 74, 76, and rotation of the third arm 54 about the longitudinal arm axis 52, as indicated by the arrow 63. The first to fourth joints 56, 58, 60, 61 may include bearings of a variety of kinds to facilitate the rotation of the first to third arms 16, 18, 54. Further, power and control signals should be transmissible across the first to fourth joints 56, 58, 60, 61 without twisting of wires such as by use of a slip ring or other means, thereby allowing powering and control of the various emitters 22 and motors (described further below). The first and second lateral axes 43, 44 are perpendicular to the longitudinal arm axis 52 and these axes are each perpendicular to the vertical axis 46. Rotation of the first to third arms 16, 18, 54 and rotation of the complete arm assembly 17 about four perpendicular axes 43, 44, 46, 52 provides for significant degrees of freedom for directing the irradiation of the emitters 22. Also, rotation of the first to third arms 16, 18, 54 about the respective axes 43, 44, 52 facilitates adapting the angle of incidence of the radiation to the aircraft surfaces. In various embodiments, one, some or all of the first to fourth joints 56, 58, 60, 61 allow rotation of at least substantially 180°, at least substantially 360° and optionally continual rotation in one or both directions. Thus, the radiation from the emitters 22 can be aimed downward towards the floor or seat bases, along the aisle towards the seat backs (front or rear sides) or upwards towards the overhead compartments 38. Yet further, the height of the first to third arms 16, 18, 54 is independently adjustable along the tower 26 along the vertical axis 46, as indicated by arrows 70, 72, thereby further increasing the adaptability of the aircraft disinfecting system 8 to the interior of the aircraft to ensure optimal angle of incidence and distance from the aircraft surfaces to ensure proper disinfecting.

In some embodiments, further degrees of articulation of the first to third arms 16, 18, 54 may be added. The first and second arms 16, 18, and also the optional third arm 54, are shown extending perpendicularly from the tower 26 relative to the vertical axis 46. This angle may also be controllable through the first to third joints 56, 58, 60 so that the first to third arms 16, 18, 54 may be angled upwardly and/or downwardly relative to the depicted perpendicular orientation.

In some embodiments, the first to third arms 16, 18, 54 are rotatable about their respective axes 43, 44, 52 manually. The height of the first to third arms 16, 18, 54 may also be adjusted along the vertical axis 46 manually. Further, the tower 26 may be manually rotated relative to the trolley 10. Manual interaction with the first to third arms 16, 18, 54 may be direct or it may be through a user interface 65 including knobs, levers or buttons and mechanical connections (e.g. gears, pulleys, cables, etc.) between the user interface 65 and the controlled components. The depicted embodiment is, however, motorized. The tower 26 and trolley 10 includes an arrangement of motors, controller 48 and power unit 14 to provide height adjustment and rotation of the first to third arms 16, 18, 54 and rotation of the tower 26 described above.

In the exemplary embodiment, a first motor assembly 28 is coupled to rotate and extend the first arm 16. A second motor assembly 30 is coupled to rotate and extend the second arm 18. A third motor 32 (e.g. a linear motor) is coupled to raise and lower the first arm 16. A fourth motor 34 (e.g. a linear motor) is coupled to raise and lower the second arm 18. A fifth motor 36 is coupled to rotate the tower 26. A sixth motor assembly 62 is coupled to rotate and extend the third arm 54. The first, second and sixth motor assemblies 28, 30, 62 may each include two motors for independent control of extension and rotation of the first to third arms 16, 18, 54. A seventh motor 64 is coupled to raise and lower the third arm 54. The first to fourth, sixth and seventh motors 28, 30, 32, 34, 62, 64 are located in the tower 26 and the fifth motor 36 is located in the trolley 10 in the depicted embodiment but alternative distributions are possible with appropriate transmission mechanisms. The controller 48 and the power unit 14 are electrically coupled with each of the first to seventh motors/motor assemblies 28, 30, 32, 34, 36, 62, 64 for independent, bi-directional operation so that the first to third arms 16, 18, 54 can be extended and retracted as desired, can be rotated in either direction as desired and the arm assembly 17 can be rotated in either direction as desired. The user interface 65 is electrically connected to the controller 48 to provide user selected inputs for controlling the height and rotation of the first to third arms 16, 18, 54 and the rotation of the arm assembly 17. The controller 48 includes a processor 50 and memory (not shown) storing computer programming instructions for processing the inputs and generating suitable control signals for executing the user selections by control of the first to seventh motors/motor assemblies 28, 30, 32, 34, 36, 62, 64.

The user interface 65 may include any known type or combination of types of electronic user interfaces. The user interface 65 may control direction and speed of the trolley 10. In such embodiments, the wheels 12 (or at least some of them) may be motorized and may include powered steering so that the user can input a speed and direction selection to move the trolley 10 up and down the aisle 42 and elsewhere within the cabin, which controls are executed through the controller 48. The user interface 65 may also allow selection of control of the first to third arms 16, 18, 54 as described above. The user interface 65 may allow selection of intensity, wavelength and mode (pulsed or continuous) of the electromagnetic radiation, as described further below. The user interface 65 may be fixed to the trolley 10 or it may be a removable device that wirelessly communicates with the controller 48. The user interface 65 could even be implemented through a user's portable electronic device such as a smart phone or a tablet, which communicates wirelessly (e.g. via a wireless internet connection, Bluetooth or other suitable wireless technology).

In the exemplary embodiment, the trolley 10 is depicted to include a power unit 14. The power unit 14 may include one or more rechargeable batteries, which provide power for the emitters 22. In another embodiment, the power unit 14 may draw power from an external source and thus may include a plug and cord. In such an embodiment, the cord may be retractable into a coil within the trolley 10. One or more rechargeable batteries are located within the trolley 10 to power the first to seventh motors/motor assemblies 28, 30, 32, 34, 36, 62, 64, the controller 48, the sensor system 98, fans, other electronics, the emitters 22, etc. These heavier components are preferentially located at the lower portion of the trolley 10 to maximize lateral anti-tip-over stability. A power cord port (not illustrated) allows plug in charging when the trolley is not in use. The batteries can be located within or attached to the trolley. In some forms, the batteries can snap on as an exterior battery pack, and the batteries can cooperate with a separate charging station. In this way, the aircraft disinfecting system 8 can be used to sanitize several aircraft in succession by simply swapping the battery pack.

In the depicted embodiment, the trolley 10 includes a sensor system 98. The sensor system 98 may include a wheel speed or trolley speed sensor. The speed sensor may operate by sensing rotation rate of the wheels 12 and known wheel dimensions. Such a speed sensor may be a magnetic or optical wheel speed sensor. The sensor system 98 may further include a camera or other optical detector to allow the controller 48 to learn geometric information about the cabin, as described below. Although the sensor system 98 is shown located in the trolley 10, the sensor system 98 may be distributed in any way between the tower 26 and the trolley 10. For example, it may be desirable to have one or more cameras located on the tower 26 to detect geometric information about the cabin (e.g. identification of surfaces that need sanitizing and spaces within which the arms 16, 18, 54 can move).

The aircraft disinfecting system 8 may operate with varying degrees of autonomy. The aircraft disinfecting system 8 is, in some embodiments, configured to raise and lower the first and second arms 16, 18 as the trolley 10 is moved along the aisle 42 so as to move over the seat backs, down into the space between neighboring seat backs from adjacent rows (closer to seat bases and the floor) and then back above the next seat back. That is, the first and second arms 16, 18 are controlled to move up and down along the vertical axis 46 to ensure that the emitters 22 are maintained relatively close to surfaces being sanitized. Further, the first and second arms 16, 18 are rotated around their respective axes 43, 44 to maintain an angle of incidence of around 90° relative to surfaces being sanitized. Thus, the first and second arms 16, 18 may be rotated so that the emitters 22 follow a cycle when passing from one row to the next row of seats 40: aimed downwards over the tops of seat backs, backwards to face the front of seat backs, downwards to face seat bases and seat floors, forwards to face the back of seat backs. Such combined rotation and up and down movement of the first and second arms 16, 18 may be implemented by cam linkages between the wheels 12 and the first and second arms 16, 18 in embodiments not including the various first to fourth motors/motor assemblies 28, 30, 32, 34. The cam linkages may be tailored to a particular aircraft design, specifically the seat pitch.

In alternative embodiments, the controller 48 is configured to automate control signals to the first to fourth motors/motor assemblies 28, 30, 32, 34 to effect the vertical and rotational movements of the first and second arms 16, 18. The pitch between rows of seats 40 could be stored in memory for different aircraft models to allow suitable generation of control signals based on sensed speed (through a sensor system 98) of the trolley 10. In other embodiments, the pitch of rows of seats could be learned by the controller 48 based on feedback from the sensor system 98. For example, the sensor system 98 might detect the pitch of seat legs, the seats 40 themselves, the gap between the seats 40 and other geometric information using a camera or other optical detector included in the sensor system 98. This learning can be stored in memory from which the controller 48 generates control signals to execute vertical and rotational movement of the arms 16, 18. In another embodiment, the sensor system 98 may allow optical detection (e.g. through plural cameras) of the cabin as the trolley 10 is moved and the controller 48 may be responsive to feedback from the sensor system 98 in real time to move the first and second arms 16, 18 (and optionally also the third arm 54) in order to optimize angle of incidence, distance between emitters 22 and surfaces being treated and coverage of aircraft surfaces by radiation according to programmed requirements. The sensor system 98 and the controller 48 may also be configured to determine the seat configuration along each row (e.g. three seats on each side of the aisle 42, one seat on one side and two on the other side) using optical recognition from which the controller 48 can determine how far to extend the first and second arms 16, 18. In other embodiments, the sensor system 98 can include proximity sensors or other optical sensors (e.g. cameras) associated with each of the first and second arms 16, 18 to allow real-time feedback to the controller 48 on the proximity of the ends (and optionally other areas) of the first and second arms 16, 18 (and optionally also the third arm 54) to aircraft surfaces to determine where to stop extension of the first and second arms 16, 18 and optionally also the third arm 54.

In embodiments, the controller 48 may have access to a database (not shown) of aircraft models providing specifications concerning the layout and geometry of the cabin for various aircraft models. This database may be stored on a memory included in the trolley 10 or may be accessed through wireless network connectivity of the controller 48, e.g. over the internet. Such specifications may include information on dimensions and locations of each seat, the overhead compartments 38, the galleys, the toilets, the cockpit, etc. Such specifications, which can be derived from Original Equipment Manufacturer (OEM) specifications, will provide the controller 48 with detailed knowledge on the aircraft surfaces for sanitization and the spaces available for the aircraft disinfecting system 8. The controller 48 can design detailed controls for extension, rotation and height of the first, second and third arms 46, 18, 54 for a thorough sanitization of practically all aircraft surfaces based on the specifications. Such an embodiment may also utilize feedback from the sensor system 98 as detailed above in order to fine control position of the first, second and third arms 16, 18, 54, particularly to account for any differences between the specification in the database and the physical aircraft cabin, e.g. because of human occupancy, removable items, etc.

In accordance with various embodiments, the emitters 22 are in the form of Light Emitting Diodes (LEDs) that emit UV light, although other UV sources are possible. LEDs allow ready control of intensity, wavelength (through control of a selection of LEDs emitting radiation at different wavelengths) and mode of the emitted radiation. In some embodiments, the emitters 22 are capable of emitting at differing wavelengths and/or at differing intensity levels. The controller 48 and any associated drive circuitry are configured to control the intensity, the wavelength and the mode of the emitted radiation. Any one of the wavelength, the intensity and the mode of radiation emission may be set based on user selection through the user interface 65. Additionally, or alternatively, setting of UV emission is controlled autonomously based on information from the sensor system 98. In one embodiment, the sensor system 98 includes a human occupancy detector for determining whether the aircraft cabin is occupied by humans. The human occupancy detector may be a combination of one or more cameras and software to recognize a signature of a human body (e.g. based on shape, thermal profile, movement, etc.). Alternatively, humans may carry a tag that is detectable by the sensor system 98 or they may be required to register with a system of the aircraft when on board and this information can be accessed by the controller 48. When human occupancy is detected, different wavelength, intensity and/or modes of UV radiation emission may be selected by the controller 48. For example, 200-220 nm may be useable in the presence of humans because a study has shown that radiation in this range is less harmful and wavelengths of 250-270 nm may be used in the absence of humans for greater germicidal efficiency.

Human occupancy/non-human occupancy settings may be selected by the controller 48 in response to human occupancy detection from the sensor system 98 or corresponding selection from the user interface 65. The emitters 22 may be of plural kinds within the first to third arms 16, 18, 54 to emit the different wavelengths by selection of LEDs capable of emitting at the desired wavelength range or the emitters 22 may be driven to emit the different wavelengths. High intensity pulses can be applied to specific areas that are at acute angles; whereas continuous radiation levels can be used at areas where incident angle is around 90 degrees. The intensity level can be user selected through the user interface 65. Additionally, or alternatively, the sensor system 98 can detect an angle of incidence with respect to an aircraft surface, e.g. through light detectors having filters that pass or block light that is not substantially reflected at 90°. Based on the intensity of the reflected light passing through the filters, a determination can be made on angle of incidence. Alternatively, the angle of incidence can be derived from knowledge of the location and angle of aircraft surfaces, which can be determined from depth imaging or from the above described database of aircraft models, and the location of the first to third arms 16, 18, 54 and the direction of aiming of the emitters 22. The intensity of radiation emitted can be controlled by having pulsed or continuous mode emissions or controlling power settings of the emitters 22. In embodiments, the pulse frequency of emitted radiation (which is related to the time period between neighboring pulses), the wavelength of each pulse, the intensity (amplitude) of each pulse and whether to apply pulsed waveforms or continuous emission can all be controlled through the controller 48 based on programming or based on input from the user interface 65.

In operation, the trolley 10 is initially stowed off the aircraft or in a compartment in the galley, with the first to third arms 16, 18, 54 retracted, and plugged into an external power source to charge the on-board batteries of the power unit 14. When ready for use, the aircraft disinfecting system 8 is unplugged and wheeled onto the aircraft in a manner similar to known food/beverage trolleys. The trolley 10 is positioned in the aisle 42 between the first row (or last row) of seats. In some embodiments, the trolley 10 is moved along the aisle 42 autonomously or by user control through the user interface 65 or by a user (who is suitably wearing protective equipment) pushing the trolley 10. In autonomous embodiments, the trolley 10 is navigated along the aisle based on feedback from cameras or proximity sensors of the sensor system 98 or based on detailed knowledge of the cabin from the database of aircraft models or a combination thereof. The first and second arms 16, 18 are extended and the third arm 54 is extended when required using the first, second and sixth motor assemblies 28, 30, 62. The degree of extension may be controlled based on selection through the user interface 65, based on input from the cameras or proximity sensors of the sensor system 98 and/or based on cabin dimensions obtained from the database of aircraft models. The length of extension of each of the first and second arms 16, 18 and third arm 54 when required may be the same or different depending on the application. The emitters 22 are powered from the power unit 14 based on a selection through the user interface 65 or through a system selection from the controller 38. The trolley 10 moves along the aisle 42 and the first and second arms 16, 18 are raised and lowered along the vertical axis 46 to maintain spacing with aircraft surfaces and are rotated about the respective axes 43, 44 to maintain the angle of incidence. The trolley 10 may traverse each row of seats 40 twice to emit radiation downwardly towards the seats 40 and to emit radiation upwardly towards the overhead compartments 38. The trolley speed and the coordinated movement of the first and second arms 16, 18 may be programmed based on required disinfection rate and radiation intensity. The third arm 54 may be extended forwardly when the trolley moves along the aisle 42 to sanitize the floor of the aisle. The third arm 54 may additionally or alternatively be first extended at the end of aisle 42 to sanitize the galleys, toilets and cockpits. In this instance, the first and second arms 16, 18 may be retracted. The tower 26 may be rotated about the vertical axis 46 to maneuver the third arm 54. The third arm 54 is rotated about the longitudinal arm axis 52 to sanitize surfaces in the cabin according to sensed location of the aircraft surfaces from the sensor system 98 and/or information from the database of aircraft models or according to input from the user interface 65. The intensity, mode and/or wavelength of the emitted radiation is controlled based on user selection through the user interface 65 or based on detected human occupancy or determined angle of incidence of radiation. When treatment is completed, the first to third arms 16, 18, 54 are retracted to the stowed position. The aircraft disinfecting system 8 is placed back in storage or moved to another aircraft.

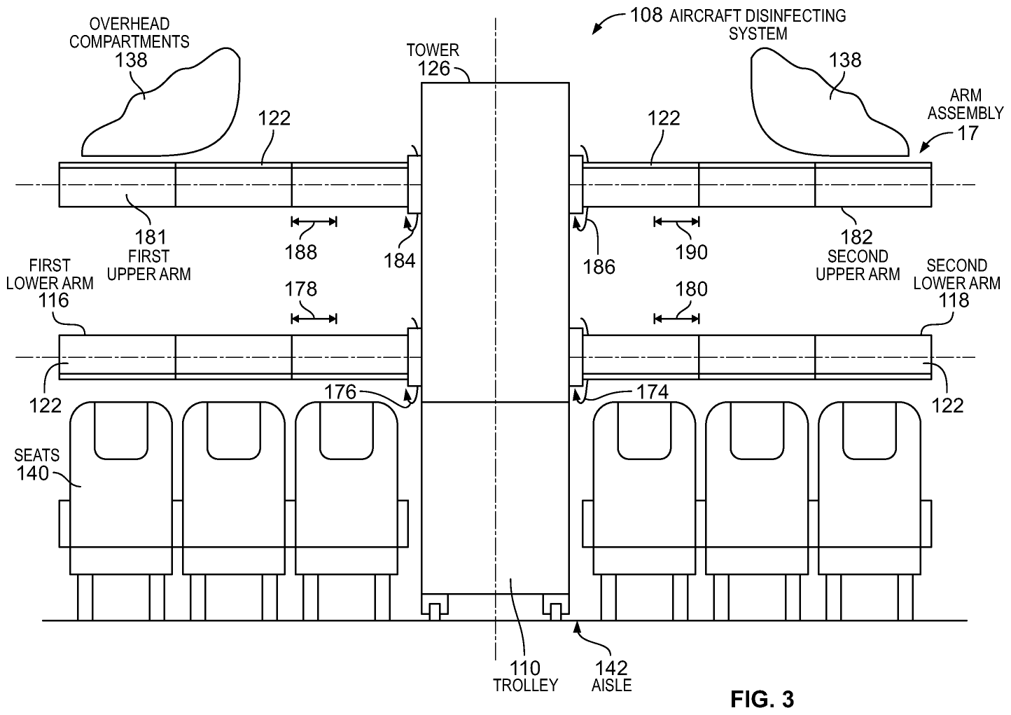

An alternative embodiment is illustrated in FIG. 3. In the exemplary embodiment of FIG. 3, the aircraft disinfecting system 108 includes bi-level arms. That is, the aircraft disinfecting system 108 includes a first lower arm 116, a second lower arm 118, a first upper arm 181 and a second upper arm 182. The first lower arm 116 and the first upper arm 181 extends over seats on one side of the aisle 142 and the second lower arm 118 and the second upper arm 182 extends over one or more seats on the other side of the aisle 142. The arms 116, 118, 181, 182 have the capabilities of the first and second arms 16, 18 described with respect to the embodiment of FIGS. 1 and 2. That is, the arms 116, 118, 181, 182 are each independently extendable to different lengths and are each independently rotatable about their own axes of extension. Each of the arms 116, 118, 181, 182 is independently height adjustable. The extension, rotation and height of the arms 116, 118, 181, 182 may be manually set or set through motorization. The motorization may be primarily controlled through a user interface or through an automated control scheme as has been described above with respect to the rotation, extension and height control of the first and second arms 16, 18 of the first embodiment. The depicted embodiment of FIG. 3 allows the overhead compartments 138 and/or the ceiling to be irradiated with germicidal radiation at the same time as the seats and/or the floor by way of first and second lower arms 116, 118 having emitters 122 that face substantially or predominantly downward (at controllable angles relative to rotation about the axes of extensions) and first and second upper arms 181, 182 having emitters 122 that face substantially or predominantly upward (at controllable angles relative to rotation about the axes of extensions). In this way, both lower seating regions and upper overhead compartment regions of the aircraft cabin can be sanitized with a single pass of the trolley 110 and the tower 126.

In an additional embodiment, the aircraft disinfecting systems 8, 108 may be modified to include a detachable, hand-held light bar to allow greater disinfection control. For example, the light bar may include UV radiation emitters and be mounted to the trolley 10, 110 in a suitable holder. The light bar may include a rechargeable battery that may be recharged when the light bar is placed in the holder (either wirelessly or through a wired charging port) or the light bar may be connected by a cord to the trolley and the power unit 14.

Those of skill in the art will appreciate that the various illustrative logical blocks, modules, circuits, and algorithm steps described in connection with the embodiments disclosed herein may be implemented as electronic hardware, computer software, or combinations of both. Some of the embodiments and implementations are described above in terms of functional and/or logical block components (or modules) and various processing steps. However, it should be appreciated that such block components (or modules) may be realized by any number of hardware, software, and/or firmware components configured to perform the specified functions. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. For example, an embodiment of a system or a component may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments described herein are merely exemplary implementations.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such that the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In this document, relational terms such as first and second, and the like may be used solely to distinguish one entity or action from another entity or action without necessarily requiring or implying any actual such relationship or order between such entities or actions. Numerical ordinals such as “first,” “second,” “third,” etc. simply denote different singles of a plurality and do not imply any order or sequence unless specifically defined by the claim language. The sequence of the text in any of the claims does not imply that process steps must be performed in a temporal or logical order according to such sequence unless it is specifically defined by the language of the claim. The process steps may be interchanged in any order without departing from the scope of the invention as long as such an interchange does not contradict the claim language and is not logically nonsensical.

Furthermore, depending on the context, words such as “connect” or “coupled to” used in describing a relationship between different elements do not imply that a direct physical connection must be made between these elements. For example, two elements may be connected to each other physically, electronically, logically, or in any other manner, through one or more additional elements.

While at least one exemplary embodiment has been presented in the foregoing detailed description of the invention, it should be appreciated that a vast number of variations exist. For example, the solution can be further broadened to non-weather information (e.g. airspaces). It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. An aircraft disinfecting system, comprising:
a trolley that can move along an aisle of an aircraft;
an arm assembly coupled to the trolley and comprising at least first and second arms that can extend over one or more seats on either side of the aisle along respective axes of extension; and
an electromagnetic radiation apparatus coupled to the trolley and the arm assembly for disinfecting, by electromagnetic irradiation, surfaces associated with the seats;
wherein the first and second arms are rotatable about the axes of extension, respectively.

2. The aircraft disinfecting system of claim 1, wherein the electromagnetic irradiation is ultraviolet (UV) irradiation.

3. The aircraft disinfecting system of claim 1, comprising a controller configured to adjust the wavelength of the electromagnetic irradiation.

4. The aircraft disinfecting system of claim 3, wherein the controller is configured to select the wavelength of the electromagnetic irradiation between a first range of 250 to 270 nm and a second range of 200 to 220 nm.

5. The aircraft disinfecting system of claim 3, wherein the controller is configured to adjust the wavelength of the electromagnetic radiation in response to an input representing human occupancy in the aircraft.

6. The aircraft disinfecting system of claim 1, comprising a controller configured to adjust an intensity of the electromagnetic irradiation.

7. The aircraft disinfecting system of claim 6, wherein the controller is configured to emit the electromagnetic radiation in a pulsed or continuous mode.

8. The aircraft disinfecting system of claim 6, wherein the controller is configured to adjust the intensity of the electromagnetic irradiation in response to a user selection.

9. The aircraft disinfecting system of claim 6, wherein the controller is configured to adjust the intensity of the electromagnetic irradiation in response to a sensed location of the trolley.

10. The aircraft disinfecting system of claim 1, wherein the electromagnetic radiation apparatus is configured to emit the electromagnetic radiation at different selectable wavelengths and with selectable radiation doses.

11. The aircraft disinfecting system of claim 1, wherein the arm assembly comprises a third arm that can extend forwardly.

12. The aircraft disinfecting system of claim 1, wherein the arm assembly is rotatable relative to the trolley about a vertical axis extending perpendicular to the horizontal plane defined by the aisle.

13. The aircraft disinfecting system of claim 1, wherein the arm assembly is height adjustable relative to the trolley.

14. The aircraft disinfecting system of claim 13, wherein the arm assembly is automatically height adjustable relative to the trolley as the trolley proceeds along the aisle.

15. The aircraft disinfecting system of claim 14, comprising a cam assembly coupled between wheels of the trolley and the arm assembly for height adjusting the arm assembly as the trolley proceeds along the aisle.

16. The aircraft disinfecting system of claim 14, comprising a sensor for sensing a position of the trolley along the aisle and an automated mechanism for height adjusting the arm assembly based on the sensed position.

17. The aircraft disinfecting system of claim 1, wherein the first and second arms are independently adjustable from one another along a length and/or in a height direction from the trolley

18. The aircraft disinfecting system of claim 1, wherein the first and second arms are adjustable relative to the trolley in an angular direction relative to a horizonal plane defined by the aisle.

19. The aircraft disinfecting system of claim 1, wherein the arm assembly is height adjustable relative to the trolley and wherein the first and second arms are rotatable relative to the trolley about the axes of extension, respectively, to change an angle of incidence of the electromagnetic irradiation, and comprising a controller configured to automate the height adjustment of the arm assembly and the rotation of the first and second arms.

20. The aircraft disinfecting system of claim 1, wherein the arm assembly includes a plurality of arms for extension over one or more seats on one side of the aisle and a plurality of arms for extension over one or more seats on the other side of the aisle.
